(19) United States
(12) Patent Application Publication
(10) Pub. No.: US 2011/0208284 A1
(43) Pub. Date: Aug. 25, 2011

(54) PROTECTIVE SLEEVE FOR A MEDICAL DEVICE, SYSTEM COMPRISING A PROTECTIVE SLEEVE AND A MEDICAL DEVICE, AND A METHOD FOR THE PRODUCTION THEREOF

(75) Inventors: Andreas Hofmann, Graefenberg (DE); Michael Filippi, Zuerich (CH); Raimund Moehl, Forch (CH); Helen Strub, Rotkreuz (CH)

(73) Assignee: BIOTRONIK AG, Buelach (CH)

(21) Appl. No.: 13/029,461

(22) Filed: Feb. 17, 2011

Related U.S. Application Data
(60) Provisional application No. 61/307,892, filed on Feb. 25, 2010.

(51) Int. Cl. A61M 25/00; A61F 2/84; A61M 25/10; B23P 17/04; B23P 25/00

(52) U.S. Cl. 623/1.11; 604/528; 604/103.05; 29/428; 29/458

(57) ABSTRACT

A protective sleeve for a medical device including a main body which has a distal end and a proximal end and on the lateral surface of which at least one stop element is disposed, which during insertion of the protective sleeve into an introducer limits the insertion depth of the protective sleeve into the introducer and which preferably is disposed at the distal end of the main body.

### CROSS REFERENCE TO RELATED APPLICATIONS

This invention claims benefit of priority to U.S. patent application Ser. No. 61/307,892, filed on Feb. 25, 2010; the contents of which are herein incorporated by reference in their entirety.

### TECHNICAL FIELD

The present invention relates to a protective sleeve for a medical device, to a system comprising a medical device and a protective sleeve, and further to a method for producing and handling such a system.

### BACKGROUND OF THE INVENTION

Protective sleeves for medical devices are typically intended to protect a medical device during transport. A sleeve shall be understood as a hollow body, such as a hollow cylinder, hollow prism or tube, which can be slid over an elongated object, such as a tube, a wire, a pin or the like, thereby providing a protective or insulating function, for example. Medical devices protected by such a protective sleeve include, for example, devices for minimally invasive surgery, in particular catheters or implants, such as stents.

A protective or insulating function for a medical device is required, in particular, for the internal part of a medical device. The internal part of a medical device shall be understood as the part which is inserted entirely or partially into a human or animal body. The external part, in contrast, is the part of a medical device which is not inserted into the body. In the case of a catheter, this can be the shaft.

In general, small tubes or hoses having different diameters and made of different materials, which can be used to probe, empty, fill or rinse hollow organs, such as the bladder, stomach, intestine, blood vessels or the heart, are referred to as catheters. A special form of catheter is the balloon catheter, which is used, for example, in percutaneous transluminal coronary angioplasty (PTCA). When using this technique, a constricted vessel, such as a coronary vessel, is expanded from the inside by inflating a balloon (balloon dilatation).

These days, stents are employed especially frequently as implants for minimally invasive surgery and are used for the treatment of stenoses (vascular constrictions). They have a body in the shape of an optionally perforated tubular or hollow-cylindrical mesh structure, which is open at both longitudinal ends. The tubular mesh of such an endoprosthesis is usually inserted into the vessel requiring treatment by way of a catheter and is intended to support and/or permanently dilate the vessel. In order to achieve dilatation of a vessel, stents are arranged, for example, on the balloon of a balloon catheter. As the balloon is inflated, the vessel and the stent are dilated at the same time.

In order to be able to insert the internal part of a medical device into the desired hollow organ, generally an introducer (also referred to as a probe guide or sheath introducer) is placed in the body or disposed on the body. The introducer has one or more access openings for inserting the medical device or devices.

U.S. Pat. No. 7,104,981 B2 describes an apparatus and a method for inserting a catheter into the aorta. Here, an introducer is inserted into a blood vessel and fixed to the body. Catheters having different shapes and functions can be inserted into the blood vessel through the introducer, which proximally is provided with two access openings, without having to set up a new access to the blood vessel during each catheterization. The respective catheter is directly inserted into one of the access openings of the introducer.

Medical devices such as catheters are generally stored in a transport sleeve packaged in a sterile manner. The transport sleeve serves to provide mechanical stabilization and to protect the medical device. For the intended use of conventional medical devices, the transport sleeve must first be removed from the device, for example by pulling it off a catheter in the forward direction. Depending on the type and shape of the medical device, there is the disadvantage that the medical device, after having pulled off the transport sleeve, is no longer supported thereby and may become deformed, such as sag, due to the flexibility thereof. This applies in particular to flexible balloon catheters.

In order to insert the medical device removed from the transport sleeve into the introducer so that it can be inserted through the same into a hollow organ of the body, the user (for example, medical staff or a specialized physician) must grab it and manually insert it into the introducer. Depending on how flexible the medical device is, insertion into the introducer may be difficult. For example, a catheter must be inserted with great caution so as not to damage the catheter. However, frequently there is no time to operate with the necessary caution, for example in medical emergencies.

A further disadvantage is the fact that medical devices, particularly stents, catheters, and in particular the balloon of a catheter, are frequently provided with a coating. When removing the transport sleeve and subsequently manually inserting the coated catheter into an introducer, the coating is subject to a variety of mechanical stresses, which can damage or even destroy it. In addition to unpacking the catheter and the user grabbing it, especially the insertion into the introducer through an appropriate seal of the access opening of the introducer should be mentioned with respect to the mechanical stress. Consequently, the catheter may incur mechanical damage or the coating may be wiped off.

### SUMMARY OF THE INVENTION

The object of the present invention is therefore to create a protective sleeve for a medical device, and a system comprising a medical device and a protective sleeve, and further a method for producing and handling a system, which overcome the disadvantages known from the prior art and better protect the medical device and the coating thereof.

The object is achieved, for one, by a protective sleeve, which can be inserted into an introducer. For this purpose, the protective sleeve comprises a main body which has a distal end and a proximal end and on and/or in the lateral surface of which at least one stop element is disposed, which during the insertion of the protective sleeve into the introducer limits the insertion depth of the protective sleeve into the introducer and which preferably is disposed at the distal end of the main body.

The distal end of the protective sleeve denotes the end which is further away from the person operating the protective sleeve or the medical device disposed therein, and is inserted into an introducer, for example. In contrast, the proximal end is the end opposite of the distal end of the protective sleeve.

The advantage of the protective sleeve according to the invention is, for one, that the mechanical stress on the medical device is reduced or minimized. The user no longer must remove the protective sleeve according to the invention in order to handle the medical device and insert it into the introducer. The medical device can be inserted into the introducer together with the protective sleeve, and the further insertion of the medical device into the body to be treated can also be performed through the protective sleeve. In this way, the medical device is exposed to considerably lower mechanical stresses, such as friction or torsion. Furthermore, the risk of kinking is reduced during insertion of the medical devices into the introducer.

A further advantage of the protective sleeve according to the invention is that during handling the user no longer must come in contact with the medical device, such as a catheter or stent, but can grab it in the region of the protective sleeve, thereby preventing damage and/or contamination of the coating, which, for example, may comprise a pharmaceutically active substance. This also creates the advantage that the direct contact of the user with the coating of the medical device is avoided. As a result, the protective sleeve described not only prevents the premature loss of the coating, but also protects the user, which is to say the physician or other medical staff, from exposure to the coating, which may comprise a pharmaceutically active substance (such as a cytotoxic substance).

A particular advantage is provided by the stop element, which prevents the protective sleeve from being accidentally inserted too deep into the introducer. As a result, the protective sleeve cannot become jammed or canted. The stop element also ensures that a seal disposed at the opening of the introducer can be avoided. When inserting a catheter into an introducer in the conventional manner, the sealing membrane must be penetrated directly by the catheter, thereby generating considerable friction of the membrane at the catheter.

The protective sleeve according to the invention is suited in particular as an insertion aid and as protection for a balloon catheter, which in particular may have a coating, preferably comprising a pharmaceutically active substance, on the balloon. The protective sleeve according to the invention is suited in particular as an insertion aid for a stent, which likewise may comprise a coating, preferably comprising a pharmaceutically active substance.

In a preferred embodiment, the at least one stop element of the protective sleeve can be configured as a ring or ring section, which is disposed on the lateral surface of the main body. As an alternative, the at least one stop element can be configured as a rib.

The stop element does not have to surround the main body completely, but can also be disposed only on part of the periphery of the main body. The ring or ring section typically has the largest extension transversely with respect to the longitudinal direction, while ribs, for example, can extend in the longitudinal direction on or in the lateral surface. In general, the stop element increases the diameter or the cross-section of the protective sleeve in the section in which the stop element is provided. Frequently, the stop element forms a stop surface extending substantially in the radial direction, this stop surface interacting with a corresponding stop surface of the introducer in order to limit further insertion of the protective sleeve into the introducer. The interaction includes, for example, that the two stop surfaces bear on one another.

As an alternative, the stop element can be configured in the shape of a truncated cone, wherein the smaller diameter of the truncated cone-shaped stop element is disposed toward the distal end of the main body. During insertion of the protective sleeve into the introducer, the sleeve is inserted into the introducer until the stop element, due to the conical shape thereof, bears on the inside diameter of the introducer and prevents further insertion. Due to the conical design of the stop element, limiting the insertion depth of the protective sleeve is not dependent on the presence of a stop surface in the introducer.

Advantageously, the protective sleeve has at least one predetermined breaking point, which extends in the longitudinal direction of the protective sleeve. The predetermined breaking point can be configured as a continuous slot and/or a continuous perforation, which can also extend over the entire length of the protective sleeve. The predetermined breaking point facilitates severing and removing the protective sleeve from the medical device or the shaft thereof. Again, mechanical stress and therefore potential damage of the medical device or the shaft thereof are prevented.

At the distal end and/or proximal end, the main body of the protective sleeve can preferably have a substantially conically extending section. The substantially conical section at the proximal end allows the protective sleeve to be easily slid onto the medical device, without damaging the medical device or the coating thereof. The substantially conical section at the distal end prevents damage of the medical device or the coating thereof in the event that the protective sleeve is displaced distally or that the medical device is pulled toward proximal, and in addition, it facilitates sliding the protective sleeve onto the medical device. Furthermore, the conical section at the distal end may also act as a stop element.

If conical sections are provided at both ends of the protective sleeve, they can have maximum diameters that are the same size or different from each other.

The substantially conical sections can have different shapes. They may have straight walls. The cone angles with respect to the longitudinal axis preferably range between 20° and 70°, with angles between 40° and 60° being particularly preferred. In addition, the walls of the substantially conical sections may be curved outwardly or inwardly in some sections, wherein the radius of curvature and the direction of curvature will depend on the respective application purpose of the protective sleeve.

In a refinement of the protective sleeve, the main body comprises a section having a reduced diameter, in particular in the direction of the proximal end of the main body. This section having the reduced diameter (taper) results in clamping between the main body of the protective sleeve and the shaft of the medical device. In this way, the resulting friction is focused on the shaft, and the coating of the medical device is given additional protection, as the friction thereof on the main body of the protective sleeve can be further reduced.

The distal end of the main body of the protective sleeve is preferably closed by a film or foil, which is designed so that it can be penetrated or resealed after opening. In this way, the penetration of foreign objects and/or contaminations into the protective sleeve is prevented, which could impair the medical device or the coating thereof. The film or the foil can be pulled off by the user directly before handling the protective sleeve, which is to say before insertion into the introducer, so as not to block the path of the medical device. The film or foil may also be designed so thin that the medical device can penetrate it without becoming damaged, after the protective sleeve has been inserted into the access opening of the introducer. In this way, the medical device can be inserted from the protective sleeve into the introducer without notable resistance. The film or the foil is advantageously so thin that it cannot damage the coating comprising a pharmaceutically active substance on the medical device.

In a further preferred embodiment, the inner surface of the main body of the protective sleeve is provided with a non-stick coating and/or a coating enhancing the gliding properties, wherein the coating preferably comprises a compound from the group consisting of PTFE (polytetrafluoroethylene) and silicone. The advantage of the coating is that the medical device and/or the coating thereof cannot adhere to the inner surface of the protective sleeve during storage and transport. Furthermore, the medical device can glide better in or through the introducer when inserted therein. In this way, a coating that may be present on the medical device remains intact.

For visual and/or hygienic and/or handling reasons, the protective sleeve according to the invention may also be coated on the outer surface thereof.

The dimensions of the protective sleeve depend on the application purpose and the medical devices to be used, or the insertion opening of the introducer. Advantageously, due to the shape of the hollow organs to be examined, these medical devices are designed to be substantially round or oval, so that the inner surface of the protective sleeve preferably has a round or oval cross-section. However, the protective sleeve, and consequently the cross-section of the inner surface thereof, can also be configured in a polygonal shape. Particularly preferred sizes of the protective sleeve include lengths between 50 mm and 200 mm, with outside diameters of the main body ranging between 2.2 mm and 2.6 mm and inside diameters of the main body ranging between 1.6 mm and 2.0 mm. The stop element has a length (in the longitudinal direction) of 5 mm to 15 mm, preferably 8 mm to 12 mm.

The main body of the protective sleeve according to the invention may comprise a weldable plastic, preferably HDPE (high-density polyethylene), and the stop element may comprise a plastic material which can be bonded to this weldable plastic, preferably a polyether-polyamide block copolymer (such as Pebax®).

The material for the main body, preferably a plastic material, must meet a variety of requirements. For example, preferably it should be weldable in order to ensure good processing. In addition, the material must be suited for sterilization and/or disinfection employing commercially available reagents and/or methods. In addition, the material used must have sufficient mechanical stability in order to effectively protect the medical device. The preferred material used for the main body is therefore HDPE. However, as an alternative, or in addition, polyamide or a polyamide block copolymer can also be used.

The stop element is produced of a material which can be bonded to the material of the main body. Preferably, the material is a polyamide block copolymer which is applied onto the main body. The element may be harder than the main body, which is to say it may have a higher Shore D hardness.

As an alternative, the stop element may be integrally formed with the main body. This means that either the main body and the stop element are molded together from the same material, or the main body and the stop element are produced from different materials, wherein, for example, first the stop element is produced in the form of the protective sleeve and then encapsulated with the material of the main body. Furthermore, the protective sleeve can be extruded together with the main body and the stop element. In a further embodiment, the protective sleeve can also be produced from a hose, in that a hose having the outside diameter of the stop element is used and the remaining region of the main body is subsequently calibrated. In this context, calibration shall be understood as a forming method, during which the dimensions of the hose in the starting size are reduced to the desired dimensions by way of tensile forces.

The protective sleeve according to the invention is designed as a throw-away article for hygiene reasons and practical aspects. However, it is also possible to reuse the protective sleeve after sterilization and/or disinfection, if the material and shape are selected appropriately.

The object is furthermore achieved by a system which comprises a medical device, particularly a catheter and/or a stent, and a claimed protective sleeve according to one of the above embodiments, wherein the protective sleeve surrounds the medical device in the longitudinal direction at least in one section of the extension of the medical device. In this system, the medical device is preferably designed as a balloon catheter and the protective sleeve, in this example, surrounds the balloon catheter at least in the section in which the balloon is disposed.

The system according to the invention reliably protects the medical device from damage and/or contamination. In the preferred embodiment, in which the medical device is a balloon catheter, the balloon, the pleats thereof, and optionally the coating comprising at least one pharmaceutically active substance, are protected. Analogously, the stent and/or the optionally present coating thereof are protected by a pharmaceutically active substance. In other respects, the system according to the invention has the same advantages as the protective sleeve described above. In addition, in the system according to the invention the protective sleeve can function at the same time as a transport sleeve for the medical device to be used.

As was already described above, catheters are small tubes or hoses having different diameters. Balloon catheters are used primarily in angioplasty for dilating or reopening a vessel. First, a guide wire is introduced into the vessel to be treated and then the balloon catheter, which is composed of a hose comprising an undilated balloon in a predefined region along the hose, is pushed forward along the guide wire to the site of the vessel to be treated, so that the balloon is placed in the region of the vascular site to be treated, which has a stenosis, for example. Thereafter, the balloon is dilated, which is to say inflated and/or expanded, thereby reopening or dilating the site to be treated. Finally, the balloon is deflated again and removed from the vessel along the guide wire. At the same time, or subsequent thereto, the guide wire is also pulled out of the vessel. As a result of the dilatation or reopening of the vessel, the flow of body fluid in the vessel is no longer impaired, or no longer impaired to the previous extent. Furthermore, a stent can be inserted into the particular vascular section, for example, using the catheter, wherein in this case the stent is dilated together with the balloon.

A “pharmaceutically active substance” (or therapeutically active or effective substance) in the context of the invention shall be a plant, animal or synthetic active substance (drug) or a hormone, which in a suitable dose is used as a therapeutic agent for influencing states or functions of the body, for substituting active substances produced naturally by the human or animal body, such as insulin, and for eliminating, or rendering harmless, pathogens, tumors, cancer cells or substances foreign to the body. The release of the substance has a positive effect on the healing process or counteracts pathological changes of the tissue as a result of the surgical procedure, or in oncology is used to render diseases cells harmless.

Such pharmaceutically active substances, for example, have an anti-inflammatory and/or antiproliferative and/or spasmolytic effect, whereby, for example, residual stenoses, inflammations or (vascular) spasms can be avoided. In particularly preferred embodiments, such substances may comprise one or more substances of the active substances groups consisting of the calcium channel blockers, lipid regulators (such as fibrates), immunosuppressants, calcinurin inhibitors (such as tactrolimus), antiphlogistics (such as cortisone or dichlofenac), anti-inflammatory agents (such as imidazoles), anti-allergic drugs, oligonucleotides (such as dODN), estrogens (such as genistein), endothelial forming agents (such as fibrin), steroids, proteins, hormones, insulins, cytostatic drugs, peptides, vasodilators (such as sartanes), and the antiproliferatively acting substances of the taxols or taxanes, preferably paclitaxel or sirolimus.

The above object is additionally achieved by a method for producing and handling a system comprising a medical device, preferably a catheter or a stent, and a protective sleeve, which can be inserted into an introducer, particularly according to any one of the preceding embodiments, preferably a system according to any one of the preceding embodiments. The method according to the invention comprises the following steps: a) providing a medical device; b) sliding on the protective sleeve such that it surrounds the medical device in the longitudinal direction at least in one section of the extension thereof; and c) inserting the protective sleeve with the medical device into an introducer.

The medical device can preferably be coated in step b) before sliding on the protective sleeve, the coating preferably comprising the at least one pharmaceutically active substance. It is also possible to apply a coating after step b).

In the method according to the invention, the medical device, optionally after being coated, is protected by the protective sleeve. In particular, the user no longer must remove the protective sleeve from the medical device in order to handle the same. Instead, the protective sleeve is directly partially inserted into an introducer. The medical device is inserted into the introducer out of the protective sleeve or through the protective sleeve. With respect to a membrane sealing the introducer, the medical device can therefore be inserted into the introducer without contact. In this way, the medical device is not exposed to mechanical stresses, such as friction or torsion. This has the advantage that damage to the medical device, such as abrasion, stress marks or the like, and kinking of the device are effectively prevented. Additionally, it is avoided that the coating, which comprises a pharmaceutically active substance, for example, is wiped off. Further advantages have already been explained above in connection with the protective sleeve according to the invention.

In a further embodiment of the method according to the invention, the system can be packaged between steps b) and c) and transported, wherein packaging and transporting may be conducted after the coating process.

In a refinement of the method according to the invention, the protective sleeve is inserted into the introducer until the insertion movement is limited by a stop element disposed in and/or on the lateral surface of the main body of the protective sleeve and by a stop surface of the introducer. In this refinement, the stop element, by interacting with the stop surface of the introducer, prevents the protective sleeve from accidentally being inserted too far into the introducer, thereby preventing it from becoming canted or jammed.

Further objectives, characteristics, advantages, and application possibilities of the invention will be apparent from the following description of one embodiment with reference to the figures. All characteristics described and/or illustrated, either alone or in any arbitrary combination, form the subject matter of the present invention, independently of their combination in the individual claims or dependent claims.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### BRIEF DESCRIPTION OF THE DRAWINGS

FIG. 1 is a perspective view of a first embodiment of the protective sleeve according to the invention from the side.

FIG. 2 is a perspective view of a second embodiment of the protective sleeve according to the invention from the side.

FIGS. 3A, 3B are cross-section views each of the access opening of the introducer, comprising the protective sleeve according to FIG. 1.

FIG. 4 is a cut-open illustration of the protective sleeve according to FIG. 1, in which a balloon catheter is disposed, and

FIG. 5 is a cross-section of the protective sleeve according to FIG. 2, in which a balloon catheter is disposed.

### DETAILED DESCRIPTION

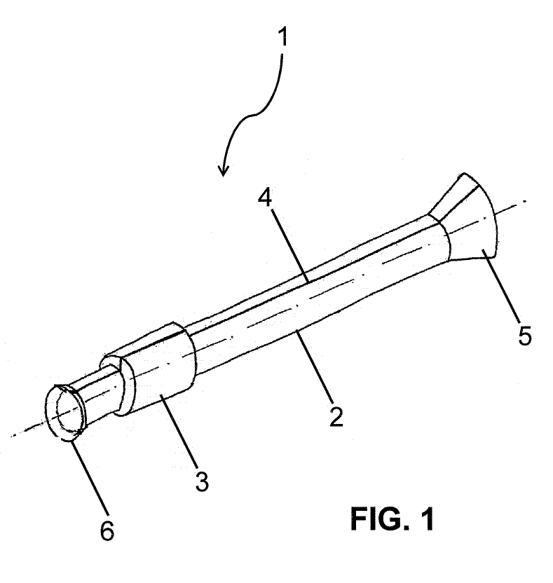

The first embodiment illustrated in FIG. 1 shows a protective sleeve 1 for a balloon catheter 10 coated with a pharmaceutically active substance. The protective sleeve 1 comprises a substantially hollow-cylindrical main body 2, on the lateral surface of which a stop element 3 is disposed.

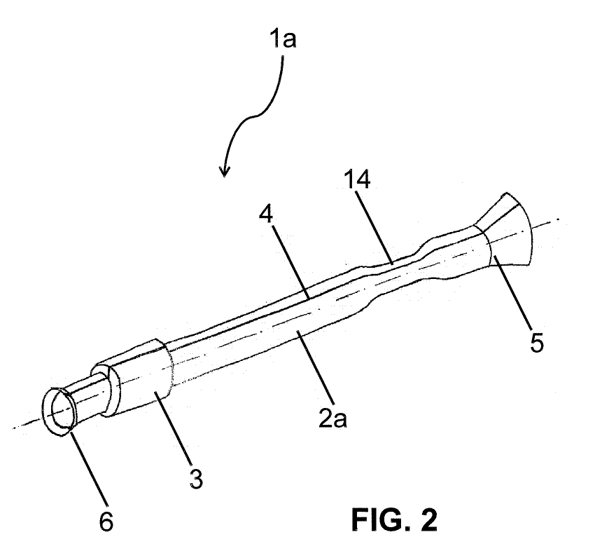

The second embodiment illustrated in FIG. 2 shows a protective sleeve 1a, which compared to the first embodiment additionally includes a section 14 having a reduced diameter (taper) toward the proximal end of the substantially hollow-cylindrical main body 2a.

The protective sleeves according to both embodiments can receive catheter sizes of up to 8 French.

The description of the further elements of the protective sleeve according to the invention refers largely to both the first and second embodiments.

The distal end of the stop element 3 is disposed at a certain distance, such as 5 mm, from the distal end of the protective sleeve 1, 1a and completely surrounds the main body 2, 2a at the lateral surface thereof. It has a larger outside diameter than the main body 2, 2a in the sections in which neither the stop element 3 nor the first cone 5 or the second 6 are disposed. The longitudinal direction of the protective sleeve 1, 1a or of the catheter 10 is marked with a dash-dotted line.

In FIGS. 1 and 2, the stop element 3 is shown as a collar or ring having a length of 10 mm, which completely surrounds the main body 2, 2a at the lateral surface thereof. As an alternative, the stop element 3 can be configured as a ring section, for example, or it may comprise a plurality of individual elements, which having the shape of a rib or ribs cover the lateral surface only partially and extend in the longitudinal direction, for example. Likewise as an alternative, the stop element 3 can be configured in the shape of a truncated cone such that the smaller diameter is disposed toward the distal end of the main body 2, 2a, The stop element 3 is rigidly connected to the main body 2, 2a or integrally formed therewith.

The maximum outside diameter of the stop element 3 depends on the inside diameter of the access opening 8 of the introducer 7, but measures 1.5 mm to 3 mm, for example.

The main body 2, 2a of the protective sleeve 1, 1a is produced using a conventional method, such as injection molding or extrusion. Materials that can be used include, for example, HDPE, PTFE, polyamide or polyamide block copolymer. In the present embodiment, the stop element 3 is preferably produced from a polyamide block copolymer. It bonds well with the HDPE of the main body 2, 2a by way of welding.

In the embodiments illustrated, a predetermined breaking point 4, having the form of a partially closed slot or a line having a smaller thickness of the respective material, runs along the entire length of the protective sleeve 1, 1a in the longitudinal direction on one side of the protective sleeve. The predetermined breaking point 4, however, can also extend along part of the length of the protective sleeve 1, 1a in the longitudinal direction. The predetermined breaking point 4 allows removal of the used protective sleeve 1, 1a from the shaft of the balloon catheter 10 in that the protective sleeve 1, 1a is severed along this point. In the regions in which the lateral surface of the protective sleeve is interrupted by a slot, the edges of the slot in the starting state are disposed so closely next to each other than the penetration of foreign objects and contaminations are effectively prevented.

The protective sleeve 1, 1a of the embodiment illustrated has a first cone 5 widening toward the outside (which is to say a substantially conical section) at the proximal end of the main body 2, 2a, and a second cone 6 widening toward the outside (which is to say a substantially conical section) at the distal end of the main body 2, 2a, The two cones 5, 6 are shown as funnels having straight walls. However, the walls can also have a curvature, at least in some sections. In the embodiments illustrated, the largest diameter of the first cone 5 is larger than the largest diameter of the second cone 6.

In the first embodiment, the protective sleeve 1 is slid with the first cone 5 onto the balloon catheter 10. In addition, the first cone 5 may enable easy sliding of the protective sleeve 1 onto a kink guard, which is not shown, but may optionally surround the balloon catheter 10 and be inserted together with the same into the patient's hollow organ. When inserting the protective sleeve 1 into the introducer 7, the second cone 6 penetrates the membrane 9 and provides good support in the membrane 9 of the introducer 7 against accidental premature removal. In the second embodiment, the protective sleeve 1a is slid onto the balloon catheter 10 from the side having the second cone 6.

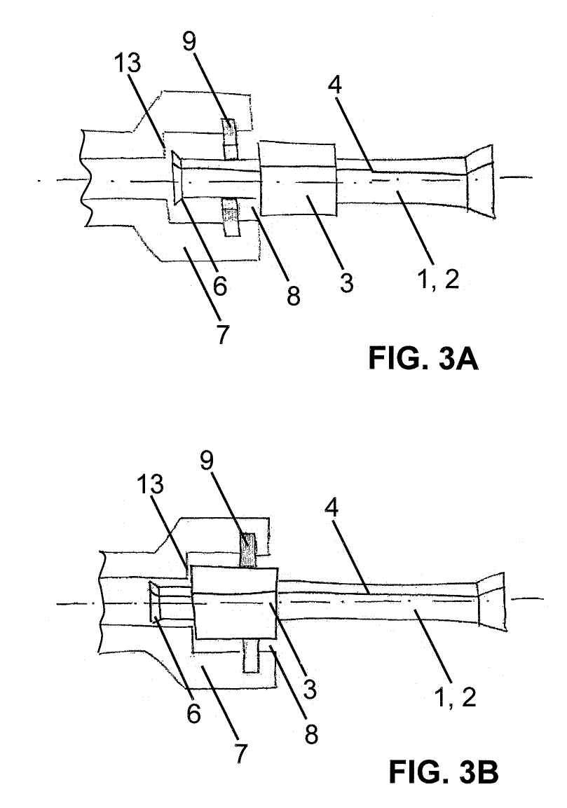

FIG. 3A is a schematic illustration of how the protective sleeve 1 is inserted into an access opening 8 of the introducer 7 with the distal end of the main body 2. For this purpose, the distal end is guided with the second cone 6 through the membrane 9 closing the access opening 8 of the introducer 7. The membrane 9, for example a septum, is produced from elastic material and forms a tight seal with the distal end of the main body 2, thereby preventing fluids or foreign matters from getting into or out of the introducer 7. In this instance, the second cone 6 with the distal end thereof forms a stop element, which bears on a stop surface 13 of the introducer 7.

FIG. 3B is a further possibility of arranging the inserted protective sleeve 1 in the introducer 7. In this configuration, the protective sleeve 1 was inserted into the access opening 8 of the introducer 7 so far that it bears on the stop surface 13 with the stop element 3 in the access opening 8 of the introducer 7. Further insertion of the protective sleeve 1 into the introducer 7 is thereby effectively prevented.

The illustrations of the protective sleeve 1 inserted into the introducer 7 shown in FIGS. 3A, 3B apply equally to the protective sleeve 1a according to FIG. 2, and for this reason a separate figure is foregone.

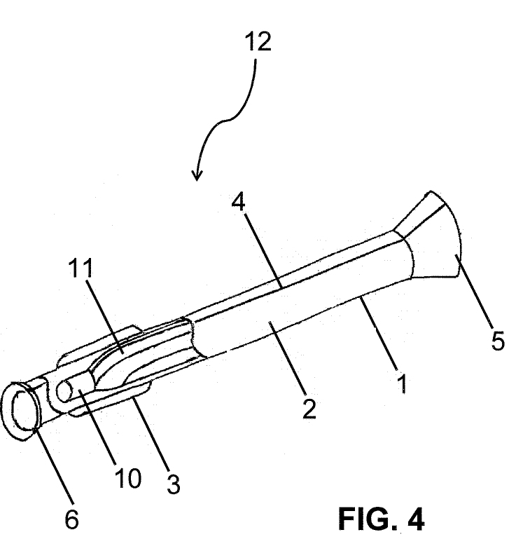

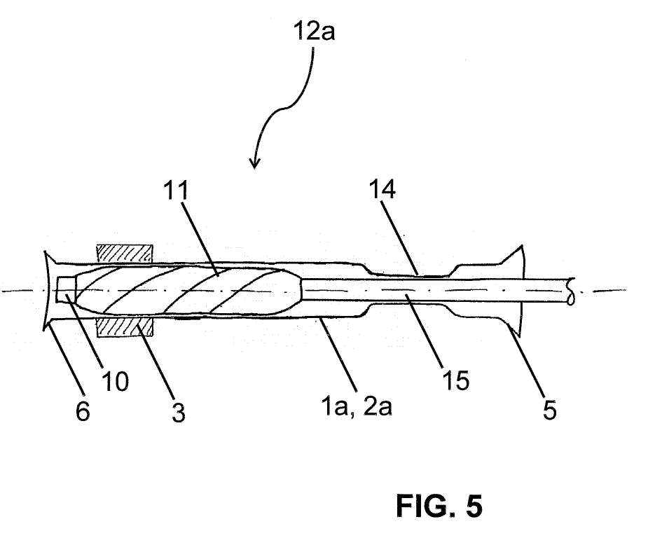

FIG. 4 is a cut-open illustration of a system 12 according to the invention, which comprises the protective sleeve 1 according to the invention and a balloon catheter 10 disposed therein. FIG. 5 shows a cross-sectional illustration of a further system 12a according to the invention, which comprises the protective sleeve 1a according to the invention and likewise a balloon catheter 10 disposed therein.

FIG. 4 does not show the shaft of the balloon catheter 10. The balloon 11 of the balloon catheter 10 preferably has a coating which comprises a pharmaceutically active substance. Balloon catheters 10 are used, for example, for percutaneous transluminal coronary angioplasty (PTCA) and percutaneous transluminal angioplasty (PTA) and in these instances are frequently coated with cytostatic drugs.

After being produced using a known method, the balloon catheter 10 is first delivered from the last production station to the coating station. On this path, the balloon catheter and the pleats of the balloon are preferably protected by a conventional protector, which is mounted at the end of the production operation. The protector is removed for the coating with a pharmaceutically active substance, for example a cytostatic drug. Thereafter, the coating with a pharmaceutically active substance and/or another compound is carried out, using known methods.

After the coating has been applied, in the first embodiment the protective sleeve 1 is carefully slid onto the coated balloon catheter 10 using the first cone 5 until the protective sleeve 1 completely surrounds the balloon 11.

In the instance of the second embodiment, after applying the coating, the protective sleeve 1a is slid carefully first onto the shaft 15 of the balloon catheter 10 using the second cone 6, and then onto the coated balloon 11, until the protective sleeve 1a completely surrounds the balloon 11. The section 14 of the main body 2a focuses the developing friction substantially on the clamping between the tapered section 14 and the shaft 15 of the balloon catheter 10, thereby further reducing the friction on the coated balloon 11.

A silicone coating, which can be disposed on the inner surface of the main body 2, 2a of the protective sleeve 1, 1a, prevents adhesion of the balloon catheter 10 and additionally reduces friction.

Subsequently, the system 12, 12a comprising the balloon catheter 10 and protective sleeve 1, 1a is packaged in a sterile manner and can be transported without difficulty.

The protective sleeve 1, 1a can, of course, also be slid onto uncoated balloon catheters 10. The protective sleeve 1, 1a then protects the thin wall of the balloon and the pleats thereof and prevents contact by the user. Optionally, a coating can also be applied onto the balloon 11 after sliding on the protective sleeve.

At the start of a medical treatment or an examination, first an introducer 7 is inserted into a venous or arterial vessel, such as in the groin, and attached to the patient. The introducer 7 is only removed at the end of the medical treatment or examination. The introducer 7 serves as a guiding device, through the interior of which the balloon catheter 10 can be inserted into the blood vessel, while sealing the puncture site at the same time.

As soon as the introducer 7 has been placed, the protective sleeve 1 can be inserted into an access opening 8 of the introducer 7. In the process, the distal end of the main body 2 of the protective sleeve 1 penetrates a membrane 9 in the access opening 8, which seals the introducer 7 with respect to the protective sleeve 1. The protective sleeve 1 can be inserted into the introducer 7 so far that it strikes against the stop surface 13 of the introducer 7 with the stop element 3.

After the protective sleeve 1 has been inserted into the introducer 7, the shaft of the balloon catheter 10 is carefully introduced further into the protective sleeve 1 from the proximal end, whereby the balloon catheter 10 with the distal end thereof penetrates a thin film, which is not illustrated, which closes the distal end of the main body 2 of the protective sleeve 1. As an alternative, the film or the foil can be pulled off immediately before inserting the protective sleeve 1 into the introducer 7, which is at the latest time possible. During the subsequent advancing of the shaft, the balloon catheter 10 is inserted entirely into the introducer 7 and further into the blood vessel.

Once the balloon catheter 10 has been inserted far enough into the vessel, the protective sleeve 1 can be removed from the catheter. For this purpose, it is first pulled out of the membrane 9 of the introducer 7, whereupon the membrane 9 closes again and immediately reseals the introducer 7. The protective sleeve 1 is now freely suspended on the shaft. The protective sleeve 1 is opened along the predetermined breaking point 4 and then bent open so far that it can be removed without damaging the shaft. During the further course of the treatment or examination, the protective sleeve 1 will then not interfere with or impair treatment.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE NUMERALS

1, 1a Protective sleeve
2, 2a Main body
3 Stop element
4 Predetermined breaking point
5 First cone
6 Second cone
7 Introducer
8 Access opening of the introducer 7
9 Sealing membrane
10 Balloon catheter
11 Balloon
12, 12a System
13 Stop surface
14 Section having reduced diameter
15 Shaft

What is claimed is:

1. A protective sleeve for a medical device, particularly for a catheter, which can be inserted into an introducer, wherein the protective sleeve comprises a main body which has a distal end and a proximal end, and in and/or on the lateral surface of which at least one stop element is disposed, which during insertion of the protective sleeve into the introducer limits the insertion depth of the protective sleeve into the introducer and which preferably is disposed at the distal end of the main body.

2. The protective sleeve according to claim 1, characterized in that the stop element is designed as a ring, ring section or truncated cone.

3. The protective sleeve according to claim 1, characterized in that the stop element is designed as a rib.

4. The protective sleeve according to claim 1, characterized in that the protective sleeve has at least one predetermined breaking point, which extends in the longitudinal direction of the protective sleeve.

5. The protective sleeve according to claim 1, characterized in that the main body at the distal end and/or proximal end has a substantially conical section.

6. The protective sleeve according to claim 1, characterized in that the main body at the proximal end has a section having a reduced diameter.

7. The protective sleeve according to claim 1, characterized in that the distal end of the main body is closed by a film or a foil, which can be penetrated or resealed after opening.

8. The protective sleeve according to claim 1, characterized in that the inner surface of the main body is provided with a non-stick coating and/or a coating enhancing the gliding properties, wherein the coating preferably comprises a compound selected from the group consisting of PTFE and silicon.

9. The protective sleeve according to claim 1, characterized in that the main body comprises a weldable plastic material, optionally HDPE, and the stop element comprises a plastic material which can be bonded to the weldable plastic, optionally polyamide or a polyether-polyamide block copolymer.

10. A system, comprising a medical device, optionally a catheter or a stent, and a protective sleeve according to claim 1, wherein the protective sleeve surrounds the medical device in the longitudinal direction at least in one section of the extension of the medical device.

11. The system according to claim 11, characterized in that the medical device is designed as a balloon catheter and the protective sleeve surrounds the balloon catheter at least in the section in which the balloon is disposed.

12. A method for producing and handling a system comprising a medical device, optionally a catheter, and a protective sleeve which can be inserted into an introducer, optionally according to the system of claim 9, comprising the following steps:
a) providing the medical device,
b) sliding on the protective sleeve such that it surrounds the medical device in the longitudinal direction at least in one section of the extension thereof, and
c) inserting the protective sleeve with the medical device into an introducer.

13. The method according to claim 12, characterized in that the system is packaged between steps b) and c) and transported.

14. The method according to claim 12, characterized in that the protective sleeve is inserted into the introducer until the insertion movement is limited by a stop element disposed on the lateral surface of the main body of the protective sleeve and a stop surface of the introducer.

15. The method according to claim 12, characterized in that the medical device is coated prior to step b), preferably using at least one pharmaceutically active substance.
